(19) United States
(12) Patent Application Publication
(10) Pub. No.: US 2009/0018151 A1
(43) Pub. Date: Jan. 15, 2009

(54) Topical Treatment of Peripheral diabetic complications

(76) Inventor: Ezekiel Fink, Los Angeles, CA (US)

Correspondence Address: Mark J. Pandiscio;Pandiscio & Pandiscio, P.C., 470 Totten Pond Road, Waltham, MA, 02451-1914, US

(21) Appl. No.: 12/070,930

(22) Filed: Feb. 23, 2008

Related U.S. Application Data
(60) Provisional application No. 60/903,041, filed on Feb. 23, 2007.

(51) Int. Cl. A61K 31/506; A61P 3/10

(52) U.S. Cl. 514/275

(57) ABSTRACT

This invention relates to agents for treating, ameliorating, or preventing diabetic complications specifically diabetic neuropathy and skin ulceration by topical application of materials containing one or more arterial or venous dilation components and one or more fast sodium channel inhibitors. Particularly, the invention relates to treating or ameliorating agents that contain minoxidil and its related congeners, which have both potassium channel agonist properties and nitric oxide agonist properties as an active ingredient and which are effective against diabetic complications such as neuropathy, peripheral circulation disorders and skin ulcerations. Unique methods of delivery including delivery through impregnated socks or gloves to match the distribution of pain are also noted.

### REFERENCE TO PENDING PRIOR PATENT APPLICATION

This patent application claims benefit of pending prior U.S. Provisional Patent Application Ser. No. 60/903,041 filed Feb. 23, 2007 by Ezekiel Fink for TOPICAL TREATMENT OF PERIPHERAL DIABETIC COMPLICATION, which patent application is hereby incorporated herein by reference.

### FIELD OF THE INVENTION

The present invention relates to compositions and methods for the treatment and prevention of peripheral neural and vascular ailments. In the methods of the present invention, a flavonoid is administered to a patient suffering from a peripheral neural or vascular ailment.

### BACKGROUND OF THE INVENTION

### A. Small Fiber Neuropathy

The phrases “peripheral neuropathies” and “small fiber neuropathies” are used interchangeably herein to refer to a set of conditions characterized by functional changes or pathological changes, or both, in the small, unmyelinated nerve fibers of the peripheral nervous system.

Peripheral or small fiber neuropathies may be caused by any of about a hundred identified factors that can produce nerve damage. The cause may be metabolic, for example hypertriglyceridemia or pellagra. Toxic exposures may also cause small fiber neuropathies, for example those resulting from alcoholism, excessive doses of vitamin B.sub.6, exposure to toxic metals such as thallium, or exposure to certain chemotherapeutic agents, such as vinca alkaloids. Certain congenital conditions, including amyloidosis, an-.alpha.-lipoproteinemia (Tangier's), and alpha.-galactosidase (Fabry's), are known to cause small fiber neuropathies. Small fiber neuropathies may result from infections such as leprosy, or diseases such as AIDS, herpes simplex, herpes zoster (shingles), cytomegalovirus, hepatitis B and C, Lyme disease, autoimmune diseases, Fabry disease, diphtheria, vasculitis, and porphyria. In approximately 15% of cases, the cause of the small fiber neuropathy cannot be determined. The neuropathy is then referred to as idiopathic.

Patients afflicted with peripheral neuropathies have pain in their extremities. The pain may at first be perceived as a tingling sensation in the fingers or toes. Decreased sensitivity to heat or cold is also a common early symptom. Frequently, however, a physical examination will show that the patient's reflexes, strength, sensory levels, and electrophysiology are normal. This has historically complicated the diagnosis of peripheral neuropathies, or led to underdiagnosis, especially in the early stages of the neuropathies. Recent technology, however, including skin biopsies and measurement of the density of different nerve fiber types in the epidermis, has improved the likelihood of detecting peripheral neuropathies.

Small fiber or peripheral neuropathies tend to progress by spreading upward, and patients may develop intense pain and/or a burning sensation that can be so severe as to be debilitating. Other symptoms of these neuropathies include cold hands or feet, cramps, muscle weakness and/or atrophy, eventual loss of perception of pressure, pain and/or temperature, neuropathic ulcers, lack of sweating, dry eyes, dry mouth, impotence, and restless leg syndrome.

In some cases, treatment of the underlying cause may also reverse or alleviate small fiber neuropathies. When the underlying cause is unidentifiable or otherwise untreatable, however, treatment consists of reducing the symptoms of the neuropathies, typically by administering medications known to decrease pain from neuropathy and related conditions. These medications include tricyclic antidepressants, anticonvulsants, opioid medications, and local anesthetics applied to the painful area. An afflicted patient may also undergo physical and occupational therapy to improve mobility and function. There are a limited number of medications that reverse and/or prevent the pathology of diabetes. These treatments are primarily focused on controlling the level of blood sugar.

Often, the symptoms of peripheral neuropathies do not vary due to their underlying causes. For example, diabetic neuropathy, a type of peripheral neuropathy, is a fairly common long-term complication of diabetes mellitus that shares many of the symptoms of peripheral neuropathies and is, therefore, included among the peripheral neuropathies as defined herein. The cause of diabetic neuropathy, however, is believed to be a chronic systemic excess of the glucose metabolite sorbitol. Further, treating the underlying cause of diabetic neuropathy, that is, improving glycemic control, will often prevent the symptoms from worsening.

### B. Diabetes

Diabetes mellitus is a group of chronic metabolic diseases characterized by high blood sugar levels, which result from defects in insulin secretion, or action, or both. Over time, diabetes can lead to damage to multiple organ systems.

Diabetic peripheral neuropathy is one of the most frequent complications of diabetes. Symptoms include numbness, tingling sensations and pain particularly in the toes or feet. This gradually evolves to loss of sensation and motor function as nerve damage progresses.

### C. Diabetic Neuropathy

Peripheral nerves are composed of large and small diameter nerve fibers. Symptoms associated with large fiber dysfunction include weakness, numbness, tingling, and loss of balance, while those associated with small fiber damage include pain, anesthesia to pin and temperature sensation, and autonomic dysfunction. The most common nerve disease associated with diabetes depends on the length of the nerves so that it first affects the longest nerves in the feet. In this type of diabetic neuropathy, small fiber nerve dysfunction may predominate. Early symptoms, which are more prominent at night, include bilateral symmetrical foot paresthesias and pain, often described as tingling, burning, prickling, shooting-pain, deep aching, pins and needles, tightness, or cold sensations. If not already present, evidence of large fiber dysfunction, such as sensory loss, numbness, tingling, and loss of coordination may appear as the neuropathy progresses. All symptoms migrate centrally over time to affect the proximal lower limbs and upper extremities. Symptoms of neuropathic pain are prominent early: typical symptoms include spontaneous pain, allodynia (pain due to a stimulus that does not normally provoke pain), hyperalgesia (an increased response to a stimulus that is normally painful), and sensitivity to cold temperatures. As these painful symptoms often result from small fiber dysfunction, patients may have accompanying abnormalities of autonomic function. Onset of pain in most forms of diabetic neuropathy has been shown to be independent of damage to large myelinated fibers and to correlate with damage to small diameter nerves (A-delta and C fibers) that transmit pain sensation.

### D. Mechanism of Diabetic Neuropathy

The molecular mechanisms of diabetic neuropathy are multifactorial and incompletely defined. The nerve is critically dependent upon oxidative metabolism. Any disruption in the supply of blood, oxygen, glucose, or other nutritional substances can lead to nerve damage. Proposed mechanisms of disruption to normal nerve function include abnormal metabolic product formation as a result of glucose accumulation in the nerve, focal tissue ischemia in sensory and autonomic nerves as a result of endoneural hypoxia, abnormally functioning tissue repair mechanisms caused by excess glucose and oxidative stress. Normalization or lessening of the disruption of the delivery of nutritional substances or lessening or alleviation of the development of abnormal metabolic products could in theory arrest or improve the symptoms and disease associated with the complications of diabetes.

Diabetic neuropathy is also known to be reversible, if good glycemic control is instituted while the condition is in its early stages. Ulcers and sores may appear on numb areas of the foot because pressure or injury goes unnoticed. Despite adequate treatment, these areas of trauma frequently become infected and this infection may spread to the bone, necessitating amputation of the leg or foot. More than 60 percent of non-traumatic lower-limb amputations in the United States occur among people with diabetes. In the period from 2000 to 2001, this translated to approximately 82,000 amputations. The American Diabetes Association estimates that there are approximately 18.3 million people with diabetes in the United States and that of those about 60 percent to 70 percent have mild to severe forms of neuropathy. According to the Centers for Disease Control, diabetes is becoming more common in the United States. From 1980 through 2002, the number of Americans with diabetes more than doubled. One of the proposed mechanisms of this process is damage to small vessels, referred to as microvascular disease, which is thought to play a central role in development and progression of neuropathy and skin ulcers

### E. Peripheral Vascular Disease

Peripheral neuropathies, including diabetic neuropathies, can also impair circulation in the affected area. Impaired circulation can adversely affect the appearance of the skin. Adverse effects on the appearance of the skin caused by radiation injury can include, for example, redness, discoloration, and dryness of the skin.

“Peripheral vascular diseases” are diseases of the blood vessels outside the heart that lead to restriction or blockage of the blood vessels. Atherosclerosis, when it affects the extremities rather than the coronary arteries, is an example of a peripheral vascular disease. Peripheral vascular diseases may also be long-term complications of other diseases, such as Raynaud's disease, Raynaud's phenomenon, hypertension, or Buerger's disease (thromboangitis obliterans).

An early symptom of peripheral vascular disease includes pain upon exercising that is relieved by rest. These diseases are progressive, however, and patients may also experience numbness, muscle weakness or pain, loss of hair on the affected extremities, cyanosis, weak or absent pulse in the affected extremities, gait abnormalities, pain when resting, skin ulcers, and, eventually gangrene. Impaired circulation caused by peripheral vascular disease can also adversely affect the appearance of the skin. Adverse effects on the appearance of the skin caused by radiation injury can include, for example, redness, discoloration, dryness of the skin.

### F. Diabetic Skin Ulceration and Skin Breakdown

A major concern in diabetes is the worsening of the health of the skin, especially in the foot, and subsequent development of diabetic foot ulcers. Foot ulcers typically occur in areas under mechanical stress (e.g., the weight-bearing areas of the foot) or as a result of poorly fitting shoes. It is important for diabetic patients to seek regular foot care as severe complications, such as an infection that requires amputation, can develop rapidly. Foot ulcers are multifactorial in origin with neuropathy, autonomic dysfunction, and vascular insufficiency all possibly contributing to this debilitating complication.

The incidence of diabetic complications increases with time, or the number of years the patient has been suffering from diabetes mellitus. For example, under normal control of blood glucose, about one half of the patients with diabetes mellitus develop retinopathy in 10 years of affliction and in 20 years, almost all cases develop retinopathy, which is currently the leading cause of blindness (in each year, about 5000 people lose vision in retinopathy). Statistically, the incidence of neuropathy is substantially the same. About 20% of the patients with diabetes develop nephropathy in 10 years and about 30% of the patients starting renal dialysis have already suffered from diabetic nephropathy, which is one of the major reasons for the initiation of dialysis (in each year, about 6000 diabetic patients start renal dialysis). From now on, increasing number of cases that start renal dialysis will be estimated to be attributable to diabetic nephropathy. Peripheral circulation disorders occur in 10-40% of diabetic patients and they tend to worsen, which is one of the major reasons for amputations of the lower extremities.

### G. Current Clinical Treatment

i. Disease Modifying Treatment for Diabetic Neuropathy

There are no treatments that can reliably reverse the damage associated with diabetic neuropathy. Treatments that do exist are aimed at prevention or mitigation of progression. Multiple studies have established a causal relationship between glycemic control and the development and progression of the microvascular complications of diabetes. Weight loss, diet, and exercise improve both cardiovascular status and diabetic control. There is some evidence that angiotensin-converting enzyme (ACE) inhibitors improve neuropathy, however, the mechanism is unclear.

ii. Symptomatic Treatment of Diabetic Neuropathy

Although diabetic neuropathy is a commonly studied condition in trials for neuropathic pain medications, there are limited options. The tricyclic antidepressants (TCAs), Cymbalta, anticonvulsants, and opioids have been found efficacious for treatment of pain associated with diabetic neuropathy in randomized controlled trials. Several topical agents have been found in limited studies to be efficacious for the treatment of pain in diabetic neuropathy including local anesthetics, and capsaicin ((8-methyl-N-vanillyl-6-nonenamide).

a. Topical Vasodilators (Isosorbide Dinitrate)

Recent data suggest that impaired nitric oxide (NO) synthesis plays an important role in the pathogenesis of painful diabetic neuropathy. Decreased NO production has been shown to contribute to a reduction in blood flow to the ischemia sensitive nerves in type 2 diabetic patients with peripheral sensory neuropathy. Furthermore, in the animal model, impaired NO generation in the nerve has been associated with hyperalgesia.

The preservation of a vasodilatory responses to nitroglycerin (which directly releases NO) in diabetic neuropathy subjects which directly releases NO, further implicates defects in NO synthesis as the cause of impaired vascular responses in diabetes. Several studies have demonstrated that topical nitroglycerin can produce local vasodilation in the feet. One study showed that topical use of isosorbide dinitrate, a potent local vasodilator which potentiates the release of NO relieved some painful sensory symptoms in a small number of diabetic patients.

b. Minoxidil (And Other Potassium Channel Openers: Pinacidil, Diazoxide)

### Minoxidil

Minoxidil (6-(1-piperidinyl)pyrimidine-2,4-diamine 3-oxide) is a potassium channel agonist which contains the chemical structure of nitric oxide, a blood vessel dilator, and may be a nitric oxide agonist. Since Minoxidil is a nitric oxide related compound it was suspected to act via activation of enzymes involved in vasodilation, however there are no reports of specific enzymatic activation to date. Recent studies suggest that Minoxidil stimulates the production of growth factors such as vascular endothelial growth factor (VEGF) in certain cells in the skin. The mechanism by which minoxidil increases the production of VEGF is not known, however. This compound has been used in the treatment of severe hypertension and alopecia

Turning back to potassium channel activators, they are drugs that open potassium channels in cells, thereby relaxing the smooth muscle to display various actions. It is known that if applied to blood vessels, potassium channel activators increase the blood circulation by relaxing the vessels (see, for example, Japanese Patent Domestic Announcement No. 501010/1988). However, it has not been known at all that potassium channel activators are effective against diabetic complications such as retinopathy, neuropathy, nephropathy, peripheral circulation disorders and skin ulcerations.

c. Topical Anesthetics

### Lidocaine

Lidocaine (2-(diethylamino)-N-(2,6-dimethylphenyl)acetamide) alters depolarization in neurons, by blocking the fast sodium (Na⁺) channels in the cell membrane. With sufficient blockade, the membrane will not depolarize, so no action potential is produced, leading to its anesthetic effects. There are several human studies demonstrating efficacy in treatment of diabetic neuropathy.

### OBJECTS OF THE INVENTION

In general, an agent that promotes increased microcirculation will be beneficial in relieving symptoms of diabetic neuropathy. Furthermore, this improvement could result in the arrest of progression and reversal of complications of diabetes. If applied early enough in the disease process, an agent that promotes increased microcirculation could prevent the development of the complications of diabetes. The application of this therapy in a topical fashion will improve patient compliance. The therapy must be delivered in a fashion that accounts for the mechanism of injury.

Decreased microcirculation is also a long-term complication of diabetes. In general, a therapy that is effective for peripheral vascular diseases will also be effective to counter decreased microcirculation caused by diabetes.

There remains a need for a treatment for small fiber neuropathies that is clinically effective when the underlying cause of the neuropathy is unknown. A need also remains for an effective treatment for small fiber neuropathies that does not suffer from the disadvantage of causing severe side effects.

In addition, there remains a need for a clinically effective treatment of peripheral vascular ailments. There is also a need to find a clinically effective therapy for skin ulcerations and associated complications of diabetes.

Accordingly, it is an object of certain embodiments of the present invention to provide a method that is effective for the treatment of small fiber neuropathies and peripheral vascular ailments.

It is another object of certain embodiments of the present invention to provide a method that is effective for the arrest of progression or reversal of the peripheral nerve and skin complications associated with diabetes.

It is another object of certain embodiments of the present invention to provide a method that is effective for the prevention of the peripheral neural and skin complications associated with diabetes.

It is another object of certain embodiments of the present invention to provide a method for the treatment of small fiber neuropathies or peripheral vascular ailments by administering a composition that does not cause severe side effects in the patient.

It is another object of certain embodiments of the present invention to provide a composition for the treatment of peripheral neuropathies or peripheral vascular ailments.

These and other objects of the present invention will be apparent from the summary and detailed descriptions of the invention that follow.

### SUMMARY OF THE INVENTION

The present invention provides a method for the treatment of disease symptoms, disease arrest, disease reversal, and disease prevention, where “disease” is defined as peripheral neural and vascular ailments associated with diabetes, by administering a composition including a therapeutically effective amount of a drug with microvascular vasodilation capability, and, optionally, an acceptable carrier.

In another embodiment, the invention relates to a composition for the treatment of disease symptoms, disease arrest, disease reversal, and disease prevention, where “disease” is defined as peripheral neural and vascular ailments associated with diabetes. The composition comprises a therapeutically effective amount of a mixture of a flavonoid having antioxidant properties, a therapeutically effective amount of a non-flavonoid antioxidant compound, and, optionally, an acceptable carrier.

### EXAMPLE

The following example is for the purpose of further illustrating the invention but are in no way taken to be meant as limiting.

Example: action of minoxidil on diabetic neuropathy. A 55 year old male subject with a history of type II diabetes and painful symmetric diabetic neuropathy with stable blood sugars for at least 4 weeks prior to beginning therapy had minoxidil applied to one foot and placebo applied to the other foot in a blinded fashion. Prior to initiating therapy, the subject was found to have significant hair loss on the feet up to the mid-calf bilaterally. He had not taken any other pain medication 3 months prior to initiating treatment and during treatment. He also had significant skin dryness. Within 2 weeks the subject reported a VAS score improvement of 3 on the foot with the minoxidil treatment. Re-growth of hair was found on the side treated with minoxidil only. Improvement of the dryness of the skin was also noted on the side treated with minoxidil only.

### DETAILED DESCRIPTION OF THE PREFERRED EMBODIMENTS

The compositions and methods of the invention provide significant, effective the treatment of symptoms, disease arrest, disease reversal, and disease prevention, where “disease” is defined as peripheral neural and vascular ailments secondary to diabetes. In addition, the composition used in the method of the invention, when administered in a therapeutically effective amount to treat peripheral neural and vascular ailments, does not cause severe side effects. One component of therapy is safe enough by FDA standards that it can be sold over the counter. The therapy can reverse the effects of peripheral neuropathy secondary to diabetes, and limit the burning sensation present with diabetic neuropathy prior to efficacy.

The topical compositions and methods of the invention also are effective in the treatment of disease symptoms, disease arrest, disease reversal, and disease prevention, where “disease” is defined as the adverse effects of the skin caused by diabetic microcirculatory complications. Adverse effects of the skin include, but are not limited to, redness, discoloration, dryness, hair loss, and formation of ulcers. Such effects, including cosmetic effects, are intended to be included within the meaning of “treating ailments.” The cosmetic effects relate to the appearance of skin in people using the composition and methods, Thus, the invention treats or cosmetically improves the appearance of persons having peripheral or neural vascular ailments by, for example, reducing or preventing redness of skin, reducing or preventing discoloration of skin, restoring hair growth, healing of ulcers, beautifying skin, improving appearance of skin, promoting attractiveness of skin, cleansing skin, removing dead or damaged skin or skin cells from skin and moisturizing skin.

The expression “therapeutically effective amount,” as used herein, refers to a nontoxic amount of a compound which is sufficient to provide the effective therapy in the treatment of disease symptoms, disease arrest, disease reversal, and disease prevention, where “disease” is defined as peripheral neural ailments, including small fiber neuropathies, and peripheral vascular complications of diabetes including but not limited to skin breakdown, ulceration and gangrene. A therapeutic amount may, for example, reduce pain, reverse sensory fiber loss or demyelination, promote angiogenesis, increase microcirculation, or increase sensory perception. The exact amount required may vary, depending on the species, age, and general condition of the patient, the nature of the complications, the particular combination of compounds, the mode of administration, and the like. The term “therapeutically” is intended to encompass beneficial cosmetic effects and effects of improved nutrition as well as medical effects. More preferred embodiments of the therapy involve minoxidil in varying topical forms applied to the area of peripheral neuropathy or skin breakdown in acceptable formulations. The therapy can be applied in existing topical formulations either through a patch that covers the area of disease. The therapy can also be applied to the affected area through topical applications including gel or foam and then covered with socks, gloves or wraps. In a third embodiment, the therapeutic minoxidil in any existing topical formulation can be impregnated in an existing sock, glove or wrap that can cover the affected area. This will increase compliance by the patient and decrease incidence of therapeutic failure.

Therapy embodiment as described above ideally would exist as minoxidil alone or as a combination of minoxidil plus sodium channel antagonist preferably lidocaine in varying dosing. The two drugs can exist in the same carrier or topical medium without reduction in therapeutic effectiveness. The therapy can be applied in existing topical formulations either through a patch that covers the area of disease. The therapy can also be applied to the affected area through topical applications including gel or foam and then covered with socks, gloves or wraps.

In a third embodiment, the therapeutic minoxidil in any existing topical formulation can be impregnated in an existing sock, glove or wrap that can cover the affected area. This will increase compliance by the patient and decrease incidence of therapeutic failure.

In another embodiment the therapy maybe centered on nitrates or NO production through topical means including but not limited to for example isosorbide nitrate. The therapy can be applied in existing topical formulations either through a patch that covers the area of disease. The therapy can also be applied to the affected area through topical applications including gel or foam and then covered with socks, gloves or wraps. In a third embodiment, the therapeutic minoxidil in any existing topical formulation can be impregnated in an existing sock, glove or wrap that can cover the affected area. This will increase compliance by the patient and decrease incidence of therapeutic failure.

Therapy embodiment as described above ideally would exist as a nitrate alone, for example, NO producer isosorbide dinitrate or a combination of a nitrate, for examples, NO producer nitrate plus sodium channel antagonist preferably lidocaine in varying dosing. The two drugs can exist in the same carrier or topical medium without reduction in therapeutic effectiveness. The therapy can be applied in existing topical formulations either through a patch that covers the area of disease. The therapy can also be applied to the affected area through topical applications including gel or foam and then covered with socks, gloves or wraps. In a third embodiment, the therapeutic minoxidil in any existing topical formulation can be impregnated in an existing sock, glove or wrap that can cover the affected area. This will increase compliance by the patient and decrease incidence of therapeutic failure.

Without wishing to be held to a particular theory, there are several physiological processes that might be affected by an effective treatment for or the prevention of neural ailments, including small fiber neuropathies. For example, an effective treatment might cause the degeneration of peripheral nerves to slow or to stop. Alternatively, an effective treatment might induce healing, or regeneration of the damaged nerves. An effective treatment might also cause the generation of new nerves to replace the damaged nerves or prevent the damage to nerves in a diabetic with an otherwise healthy nerves.

It is therefore expected that effective treatments for or the prevention of neural ailments including small fiber neuropathies will be applicable to other diseases or conditions affecting peripheral nerves. A method of regenerating nerves is beneficial to treat any patient suffering from nerve damage, for example, a skin graft patient or a victim of a nerve-severing trauma. In fact, many flavonoids are potent aldose reductase inhibitors. It has been shown that the oral administration of aldose reductase inhibitors increases the diameter of peripheral nerve bundles. Thus, it is expected that the methods of the present invention extend to the generation and regeneration of nerves.

Although the underlying cause of diabetic neuropathy is specifically known to be distinct from the other causes of small fiber neuropathies, the symptoms and pathologies are shared. It is therefore expected that an effective treatment or prevention for small fiber neuropathies will prevent, arrest, reverse, or alleviate certain symptoms of diabetic neuropathy.

The compositions used in the present invention are preferably formulated with an acceptable carrier. The non-carrier ingredients may be combined with the carrier materials to produce a particular dosage form, or be customized for a particular treatment regimen. Thus, the amount of each ingredient may vary depending on such factors as the particular mode of administration, the activity of the particular compounds employed, the age, bodyweight, general health, sex, and diet of the patient, time of administration, rate of excretion, the combination of compounds, or the severity of the illness, among other potential factors.

It is well known in the art that the individual ingredients in formulated products may interact with each other. These interactions include, for example, chemical equilibria and other chemical or physical processes. These interactions may cause the original individual components of a formulated product to change over time. Such changes may be chemical or physical. For example, an acidic component may become deprotonated in a formulation that also contains a basic component. Alternatively, one or more components may precipitate or crystallize from a formulated product. Equilibria and other processes may be expected to increase in number and complexity with increasing numbers of components in a given formulation. Such equilibria and other processes may be either innocuous or deleterious to the activity of the formulated product.

The term “stable” as used herein refers to the property of retaining at least a portion of the intended activity over a certain period of time.

The terms “mixture,” “composition” and “formulation” as used herein refer to stable mixtures, compositions, and formulations, respectively. Preferred mixtures, compositions, and formulations are stable over a period of at least about three months.

In a preferred method of the present invention, the composition is applied topically to an area of the skin in the vicinity of tissue that suffers from small fiber neuropathy or skin deterioration in order to relieve pain and other symptoms of the small fiber neuropathy or skin deterioration. The composition may also be topically applied to an area of the skin in the vicinity of tissue that suffers from small fiber neuropathy or skin deterioration in order reverse or arrest the pathology of small fiber neuropathy or skin deterioration. Alternatively, the composition may be topically applied to an area of the skin in the vicinity of tissue most likely to suffer from small fiber neuropathy or skin deterioration in order to prevent the development of skin deterioration or small fiber neuropathy.

Such areas typically include the patients' extremities, such as the fingers, toes, hands and feet, where neuropathy is often most pervasive.

Preferably, a suitable amount of the topical composition of the invention varies with the delivery method. Also preferably, a sufficient amount of the topical composition is applied to cover the afflicted area with a thin layer of the composition and the composition is rubbed into the skin until little or no residue remains on the skin. The treatment is almost immediately effective to alleviate acute symptoms, and may be continued, for a predetermined period or indefinitely, to relieve pain, arrest the disease process, reverse the pathology, prevent the return of pathology or symptoms of small fiber neuropathies or skin deterioration. The treatments also may be used, for a predetermined period or indefinitely to prevent the development of small fiber neuropathy or skin deterioration. The therapy should also provide pain relief almost immediately. The variant with minoxidil included may show relief concurrent with the return of hair growth which may or may not be a wanted side effect.

A topical formulation of the composition used in the invention preferably includes an acceptable topical carrier. Many acceptable topical carriers are known to those of skill in the art. The compounds in the composition may be dissolved, dispersed and/or suspended in the topical carrier.

Suitable hydrophilic ointment bases are known to persons skilled in the art. Exemplary hydrophilic ointment bases suitable for use in the present invention are non-U.S.P. hydrophilic ointment bases such as those made by Fougera, Inc., a division of Altana, Inc. of Melville, N.Y. Sufficient hydrophilic ointment base is employed to act as a carrier for the compounds of the composition. Typically the hydrophilic ointment base will make up more than 80% of the total composition and more preferably 80-90% of the composition is the hydrophilic ointment base. The hydrophilic ointment base functions as a carrier and preferably enhances penetration of the compounds into the skin.

One preferred topical carrier comprises hydroxymethyl cellulose. Another preferred acceptable carrier includes a solution of an acrylic copolymer in a non-aqueous solvent system. The non-aqueous solvent system preferably contains a polyethylene glycol such as, for example, methoxy polyethylene glycol 550 (MPEG). One preferred MPEG is Sentry Carbowax MPEG 550 (Dow Corp., Midland, Mich.), which is suitable for use in foods, pharmaceuticals, and cosmetics. The acrylic copolymer is preferably present in a concentration range of 3-6% by weight of solution. Also preferably, the acrylic copolymer has a molecular weight of more than 20,000. More preferably, the acrylic copolymer has a molecular weight of more than 100,000, to substantially prevent absorption of the acrylic copolymer by the human body through the skin.

Preferably, the acceptable topical carrier independently provides benefits to the patient. For example, the topical carrier may comprise panthenol or a panthenol derivative. The panthenol derivatives useful in the present invention include at least D-panthenol, DL-panthenol, and mixtures thereof. Panthenol provides skin moisturizing properties, acts as a quick, deep penetrating component of the carrier, helps deliver the compounds through the skin to the area to be treated, and may impart a healing effect to damaged tissue. The amount of panthenol or panthenol derivative preferably ranges from 0.25 to 10 weight percent, more preferably from 0.5 to 5 weight percent, and, still more preferably, from 1 to 2 weight percent, based on the total weight of the topical composition.

The topical carrier of the present invention may employ other penetrants in addition to panthenol or as an alternative to panthenol. Exemplary penetrants include ethanol, oleic acid, sodium lauryl sulfate, isopropyl myristate, glycerol monooleate, caprylic/capric triglyceride, Crodamol GTC/C, glyceryl tricaprylate/caprate, Miglyol 810, Miglyol 812, MCT oil, Neobee M5, Nesatol, oleum neutrale, oleum vegetable tenue, thin vegetable oil, light mineral oil, stearyl alcohol and lanolin mixed with suitable vegetable oils or with soft paraffin. These penetrants may have an emollient effect and facilitate the absorption of ingredients of the topical composition of the present invention into the skin.

Preferably, the topical carrier of the present invention contains at least a hydrophilic ointment base, panthenol or a panthenol derivative, and one or more dispersants, if needed to disperse insoluble or partially insoluble compounds in the carrier.

The topical carrier of the present invention may also include additional ingredients well known to persons skilled in the art, such as other carrier materials, other moisturizers, humectants, emollients, radiation blocking compounds, particularly UV-blockers, as well as other suitable materials that do not have a significant adverse effect on the activity of the topical composition in the amount used. A preferred additional ingredient for inclusion in the carrier is sodium acid phosphate, a moisturizer.

The topical composition of the present invention is preferably made by cold compounding, when one or more of the compounds employed in the topical composition are known to be sensitive to heat. Thus, in some cases, the stability or activity of the composition may be detrimentally affected as a result of other formulation methods. Preferably, a sufficient amount of the topical carrier is used, to provide a substantially homogeneous cream or ointment. It may be necessary to dissolve, disperse or suspend one or more of the ingredients prior to formulation in order to ensure substantially homogeneous distribution of one or more of the ingredients in the composition.

As noted above, dosages may vary with the manner of formulating the compounds. In general, the components of the composition, which include the flavonoid and the optional antioxidant, will make up from 0.5-90% by weight of the total composition to provide the desired daily dosage. The body weight dosages herein, when not normalized, are based on a patient having a body weight of 70 kg. The appropriate unit dosage may be determined by dividing the daily dosage by the number of unit doses per day.

### INDUSTRIAL APPLICABILITY OF THE INVENTION

The therapy used in the inventions are expected to result in an improvement in symptoms, reversal or arrest of disease process, or prevention of the diabetic complications such neuropathy, peripheral circulation disorders and skin ulcerations; theses treatments prove effective in preventing, ameliorating, alleviating and gaining recovery from various symptoms and abnormalities caused by those diseases, as exemplified by pain, numbness, intermittent claudication and gangrene.

### MODIFICATIONS OF THE PREFERRED EMBODIMENTS

It should be understood that many additional changes in the details, operation, steps and arrangements of elements, which have been herein described and illustrated in order to explain the nature of the present invention, may be made by those skilled in the art while still remaining within the principles and scope of the invention.

What is claimed is:

1. A method of treating diabetic neuropathy comprising the step of topically administering to a patient having a diabetic neuropathy a composition comprising a therapeutically effective amount of minoxidil alone.

2. A method of treating diabetic neuropathy comprising the step of topically administering to a patient having a diabetic neuropathy a composition comprising a therapeutically effective amount of minoxidil and an effective amount of one or more compounds selected from the group consisting of sodium channel blockers including but not limited to: ropivicaine, bupivicaine, lidocaine, tetracaine, etidocaine and others.

3. The method as claimed in claim 1 wherein the composition further comprises an acceptable carrier comprising a sufficient amount of a panthenol selected from D-panthenol and DL-panthenol to promote penetration of one or more compounds of the composition into the skin.

4. The method as claimed in claim 3 wherein the acceptable carrier comprises a sufficient amount of at least one non-U.S.P. hydrophilic ointment base to form a topical composition.

5. The method of claim 4 wherein the composition comprises minoxidil plus one non-U.S.P. hydrophilic ointment base to form a topical composition

6. The method of claim 4 wherein the composition comprises minoxidil plus lidocaine or related sodium channel blocker plus one non-U.S.P. hydrophilic ointment base to form a topical composition.

7. The method of claim 4 wherein the composition comprises a hydrophilic ointment base, 1 to 10 cc of DL-panthenol per pound of the composition, 1-15 mg Minoxidil (1-100%), and 1-5% Lidocaine (1-5 mg)

8. The method of claim 1 wherein the composition can be delivered in an impregnated sock or glove.

9. The method of claim 1 wherein the drug can be applied as a cream or ointment.

10. The method of claim 1 wherein the drug can be applied on a patch.

11. The method of claim 1 wherein the vasodilator can consist of a topical nitrate such as isosorbide nitrate or related nitrates that release nitric oxide.

12. The method of claim 11 can be delivered in a composition with Na channel antagonists including those listed in claim 2.

13. The method of claim 11 wherein the acceptable carrier comprises a sufficient amount of at least one non-U.S.P. hydrophilic ointment base to form a topical composition.

14. The method of claim 11 wherein the composition comprises minoxidil plus one non-U.S.P. hydrophilic ointment base to form a topical composition

15. The method of claim 1 wherein treating peripheral neural ailments comprises treating a systemic effect of the peripheral neural ailment.

16. The method of claim 1 wherein treating peripheral neural ailments comprises treating a local effect of the peripheral neural ailment.

17. The method of claim 1 wherein treating a peripheral neural ailment is selected from the group including, but not limited to decreasing pain, decreasing tingling, normalizing sensitivity to heat or cold, decreasing cramping, decreasing muscle weakness, decreasing numbness, and improving peripheral circulation.

18. The method of claim 1 wherein treatment of a peripheral nerve ailment reverses or arrests disease pathology as evidenced by, but not limited to decreasing pain, decreasing tingling, normalizing sensitivity to heat or cold, decreasing cramping, decreasing muscle weakness, decreasing numbness, improvement of hair growth or hair density, increase in size or number of blood vessels in the skin, improving peripheral circulation, normalizing of nerve fiber density, restoration of skin integrity, improvement of ulceration, or improvement of other pathology of the skin.

19. The method of claim 1 wherein treatment prevents the development of complications of diabetes including, but not limited to peripheral nerve ailment or diseases of the skin.

20. The method of claim 1 wherein treating a peripheral skin ulceration consists of restoring skin integrity, decreasing ulceration, returning color and improving peripheral circulation.
